(19) United States
(12) Patent Application Publication
(10) Pub. No.: US 2008/0138422 A1
(43) Pub. Date: Jun. 12, 2008

(54) Analgesic Compositions

(75) Inventor: John Staniforth, Wiltshire (GB)

Correspondence Address: Davidson, Davidson & Kappel, LLC, 485 7th Avenue, 14th Floor, New York, NY, 10018, US

(73) Assignee: PharmaKodex Ltd., Chippenham, Wiltshire (GB)

(21) Appl. No.: 11/883,204

(22) PCT Filed: Jan. 30, 2006
(86) PCT No.: PCT/GB06/50025

§ 371 Date: Sep. 28, 2007

(30) Foreign Application Priority Data
Jan. 28, 2005 (GB) 0501809.8

(51) Int. Cl. A61K 9/14; A61K 31/404; A61K 31/60; A61K 31/19; A61K 31/196; A61K 31/5415; A61K 31/415; A61K 31/167; A61K 31/40; A61K 31/192; A61K 31/135; A61K 31/195; A61P 25/06; A61P 25/24; A61P 25/00

(52) U.S. Cl. 424/489; 514/415; 514/159; 514/557; 514/567; 514/226.5; 514/404; 514/629; 514/423; 514/570; 514/656; 514/561

(57) ABSTRACT

The present invention relates to pharmaceutical compositions comprising an analgesic agent and providing controlled release of the agent for improved treatment and prevention of pain and pain-related conditions, such as pain-related sleep disturbance. The present invention is particularly concerned with self-administered compositions.

The present invention relates to pharmaceutical compositions comprising an analgesic agent and providing controlled release of the agent for improved treatment and prevention of pain and pain-related conditions, such as pain-related sleep disturbance. The present invention is particularly concerned with self-administered compositions.

There are a large number of compositions currently available for pain management. Where pain is particularly strong and/or persistent, these known compositions often provide inadequate relief, usually because the effect they have is not strong enough and/or because their period of their effect is too short. In both cases, there is a risk that the subject will administer an excessive dose of the analgesic composition, in an effort to find relief from the pain. This is extremely undesirable as it can lead to severe side effects and can even be fatal. Alternatively, patients and/or doctors resort to stronger analgesic agents than are necessary, in order to increase the period of pain relief and thereby reduce the dosing frequency, or to provide adequate pain relief over the entire period of treatment from a dose.

It is an object of the present invention to provide analgesic compositions which, following administration, provide long-lasting pain relief. Frequent repeated administration of doses of analgesic is inconvenient and it will usually also mean that the subject is experiencing more pain as the effect of one dose wears off and there is a delay before the next dose takes effect following administration.

It is also an object of the invention to provide analgesic compositions which have a rapid onset of effect. Not only does this reduce the time during which the subject experiences pain, but it also reduces the risk of overdosing. It is common for a subject to take a dose of an analgesic composition and, impatient for the analgesic to take effect, to then take a further dose.

Pain can have a wide variety of causes and it can also be treated by different mechanisms. It is a further object of the present invention to provide analgesic compositions which are tailored to a particular type of pain or to a particular condition, in order to provide effective analgesia.

According to a first aspect of the invention, pharmaceutical compositions are provided comprising one or more analgesic agents, wherein the release of said agent(s) is controlled provide a rapid onset of analgesic effect following administration, and preferably also delayed and/or sustained release providing an extended period of analgesia.

Preferably, the compositions of the invention are administered orally. The compositions may include two or more analgesic agents. The compositions may also include one or more further pharmaceutically active agents which provide a therapeutic effect, in addition to that of the analgesic agent.

In one embodiment of the present invention, compositions are provided which comprise an analgesic agent in a form which has a rapid onset of its analgesic effect upon administration. The agent is both rapidly released from the composition upon administration and is of a form which is rapidly absorbed and rapidly provides a therapeutic effect. In a particularly preferred embodiment, the analgesic agent is a base, for example an opioid base, such as oxycodone base, oxymorphone base, hydrocodone base, hydromorphone base, bupromorphine base or morphine base. These opioids have a very rapid onset of action.

In one of the preferred embodiments of the present invention, the compositions comprise a first analgesic agent which rapidly released and a second analgesic agent the release of which is controlled to provide a therapeutic effect until at least 8 hours after administration. The first and second analgesic agents may be the same agents or different agents. The first and second analgesic agents may be different forms of the same agent, for example with the first analgesic agent being the base form which has a rapid onset of action and the second agent being a salt form which has a slower onset of action.

The compositions release an analgesic agent rapidly, preferably so that it reaches an effective plasma concentration within a period of no more than 30 minutes, no more than 20 minutes, no more than 10 minutes, no more than 5 minutes or no more than 1 minute following administration. Preferably, the period of analgesia provided by the compositions of the present invention is at least 8 hours, more preferably at least 12 hours, and most preferably at least 24 hours.

It will clearly be desirable for the plasma concentration of the analgesic agent to be sufficient for the agent to have the desired therapeutic effect, whilst being low enough to avoid adverse side effects or to keep such side effects to a minimum.

The compositions of the present invention allow analgesic agents to be administered in reduced numbers of doses. This is not only more convenient for the patient but also reduces the frequency with which the patient experiences pain as the analgesic dose starts to wear off. Ideally, the patient will be able to take a dose of the analgesic composition of the invention at regular, predetermined intervals, without having to wait for the effect of the previous dose to wear off. This will allow the patient to avoid unnecessary exposure to pain and could mean that the patient is constantly free of pain. Each dose will preferably have an effect over a period of at least 8, at least 12 or at least 24 hours. These periods of effect mean that the doses may be conveniently taken three times, twice or once a day to provide 24-hour therapeutic effect without disruption to normal sleep patterns.

Preferably, the release of the analgesic agent from the compositions according to the present invention is controlled in order to achieve the desired release profile and plasma concentrations by employing known measures, including controlled release coatings and matrices. Such coatings and matrices are well known to the person skilled in the art. Materials commonly used to provide sustained release coatings include hydrophobic polymers such as ethylcellulose, polyvinylacetate, polymers or copolymers of acrylates or methacrylates, and mixtures thereof, and such coating materials are commercially available as Kollicoat®, Aquacoat®, Surelease®, Ethocel® and Methocel®. Materials commonly used to provide sustained release matrices in oral solid dosage forms include excipients which are hydrogel formers such as HPMC, HPC, xanthan gum, alginates, polyvinyl acetate and polyvinylpyrrolidone, and such matrix excipients are commercially available as Timerx®, Kollidon® and Methocel®.

In a preferred embodiment of the invention, the compositions comprise one or more coatings and/or excipients which control the release of the analgesic agent.

In one embodiment, the pharmaceutical composition is provided in the form of a layered oral solid dosage form. Such layered tablets are known to the skilled person. The different layers of the dosage form may provide release of the analgesic agent at different times or at different rates, using known coatings or matrices. Alternatively or additionally, the different layers may include different analgesic agents or different forms of the same analgesic agent, these different agents or forms being released at different rates or having different rates of onset of their therapeutic effect, for example as a result of differing absorption and/or degradation characteristics.

In order for the compositions according to the present invention to provide an analgesic effect over long periods of time, for example, for longer than 8 hours, the compositions may be formulated to be gastro-retentive.

Oral delivery of solid dosage forms is more convenient and accepted than other modes of administration. However, the manufacture, dispensing and administration of solid dosage forms are not without associated problems and drawbacks.

Where large doses of active agents are to be administered, tablets can become unacceptably large, being uncomfortable or impossible to swallow, especially for the elderly or the young.

There are also many patients who are unable or unwilling to take conventional orally administered dosage forms. For some patients, the perception of unacceptable taste or mouth feel of a solid dosage form leads to a gag reflex action that makes swallowing difficult or impossible. Other patients, e.g., pediatric and geriatric patients, find it difficult to ingest typical solid oral dosage forms, for example due to their size.

Other patients, particularly elderly patients, have conditions such as achlorhydria which hinder the successful use of oral solid dosage forms. Achlorhydria is a condition wherein there is an abnormal deficiency or absence of free hydrochloric acid in the gastric secretions of the stomach. This condition hinders the disintegration and/or dissolution of oral solid dosage forms, particularly dosage forms with large or insoluble excipient components.

Due to the disadvantages of the drug delivery methods discussed above, the compositions of the present invention may be prepared in multiparticulate form, such as the compositions disclosed in International Publication No. WO 03/020241. Preferably, the compositions according to the present invention are pharmaceutical compositions for gastrointestinal deposition and comprise a non-compressed, free-flowing plurality of particles comprising the analgesic agent and a pharmaceutically acceptable carrier. Preferably, the particles have a mean diameter of greater than 10 μm, greater than 20, 50 or 100 μm.

Preferably, the particles of the invention comprise at least about 40, 50, 60, 70, 80 or 90% by weight analgesic agent.

Preferably, greater than about 80, 85, 90, 95, 96, 97, 98 or 99% of a dose of the multiparticulate composition of the present invention is deposited in the gastrointestinal tract.

In some embodiments, the composition comprises two or more populations of particles, each population releasing an analgesic agent at different rates to produce different release profiles. In other embodiments, the composition includes just one population of particles, all of which are essentially the same and provide the same desired release profile.

A dose of multiparticulate composition according to the present invention is preferably from about 0.01 mg to about 1.5 g in weight, depending on the dose of the analgesic agent being delivered. Preferably, the dose is from about 1 mg to about 100 mg, or from about 10 mg to about 50 mg.

Preferably, the dose is administered to the tongue, most preferably towards the front of the tongue behind the teeth, where it can be easily swallowed with or without the need for an additional fluid. However, the invention does contemplate delivery to any portion of the tongue, taking into account, e.g., the taste sensations of different sections of the tongue and/or individual patient preference associated with comfort, e.g. mouth position.

Preferably, the mean diameter of the particles is of a size which minimizes their capacity to be inhaled into the lower lung. Typically, the mean particle size of the particles (or agglomerates) is greater than 10 μm, preferably greater than about 50 μm or greater than about 75 μm. In certain embodiments of the invention, the mean particle size range of the particles is from about 100 μm to about 1 mm, preferably from about 50 μm to about 500 μm. The particles referred to here may be granulated particles made up of smaller particles or agglomerates of smaller particles. These smaller particles are preferably nanoparticles, with a diameter of between approximately 10 nanometers and approximately 1 micron, and more preferably with a diameter of approximately 100 nanometers. The use of such small particles in the compositions of the present invention is particularly attractive where the particles in question comprise an active agent which is poorly soluble in water, such as oxycodone base.

In preferred embodiments, greater than 80% of the particles have the above disclosed diameter (not mean diameter), e.g. 80% of the drug particles have a diameter of greater than 10 μm, or a diameter of from about 100 μm to about 1 mm. In other embodiments, greater than about 90% of the drug particles have the above disclosed diameter.

Preferably, the multiparticulates comprise a pharmaceutically acceptable excipient.

The excipient preferably does not comprise more than about 60% by weight of the composition, more preferably not more than about 50%, more preferably not more than about 40%, more preferably not more than about 20%, and most preferably not more than about 10% by weight of the composition.

Fast melt compositions are known and they are typically in the form of tablets or lozenges that dissolve or disperse in a patient's mouth within a minute without the need of water or chewing. Drug delivery compositions which exhibit fast melt properties can improve patient compliance due to the ease of swallowing as well as the absence of a need for the co-administration of water or another fluid. Further, fast melt systems can be formulated so as to have a superior taste and improved accuracy of dosing as compared to liquid preparations.

Fast melt drug multiparticulate compositions have been developed to facilitate the oral administration of oral agents to patients normally having difficulty ingesting conventional solid oral dosage forms, and such compositions are, for example, disclosed in International Publication No. WO 03/074029.

In one embodiment of the present invention, the compositions are fast melt compositions. Preferably, the compositions of the present invention are multiparticulate and combine the benefits of the free flowing multiparticulate compositions described above with those of fast melt drug compositions.

Thus, in a preferred embodiment of the present invention the multiparticulate pharmaceutical compositions described above further comprise a water-soluble excipient and the composition is capable of dissolving or dispersing in a subject's mouth within 1 minute after ingestion without the co-administration of a fluid.

Preferably, the water-soluble excipient has a negative heat of solution. A significant advantage associated with such excipients, when administered via the oral cavity, is that the local cooling caused by the water-soluble excipient dissolving in saliva serves to mask the taste of the active agent in a manner which does not delay the release, or dissolution of the active agent itself.

Both non-fast melt and fast melt multiparticulate compositions according to the present invention are preferably arranged for direct, un-encapsulated administration to a patient's oral cavity. It is also preferred for the particles to be non-compressed.

The multiparticulate pharmaceutical compositions (both fast melt and non-fast melt compositions) of the present invention can be formulated in order to provide the aforementioned release profile and plasma concentrations by employing controlled release coatings and/or matrices. The skilled person would be aware of which materials to use and how much of them to use in order to prepare particles which release their payload of analgesic agent in the desired manner.

In one embodiment, the fast melt multiparticulate compositions have at least two coatings, a water-soluble excipient coating and a delayed release excipient coating. Preferably the water-soluble coating is the outermost coating. The analgesic agent which is to provide the rapid effect upon administration may be incorporated in the water-soluble coating or another rapidly soluble component of the composition.

A wide variety of analgesic agents may be incorporated into the compositions of the present invention. These include agents which have an analgesic effect but which are not normally classified as analgesics, such as those agents which are used to treat chronic or neuropathic pain, like tricyclic antidepressants, anticonvulsants and anti-migraine agents.

Suitable analgesic agents include opioid analgesics such as anileridine, buprenorphine, butorphanol, codeine, dextromoramide, dextropropoxyphene, diamorphine, dihydrocodeine, dipipanone, fentanyl, hydrocodone, hydromorphone, levorphanol, loperamide, nalbuphine, naloxone, meperidine, methadone, morphine, oxycodone, papavereturn, pethidine, phenazocine, pholcodeine, propoxyphene, tramadol, analgesics such as aspirin and other salicylates, clonidine, codine, coproxamol, ergotamine, gabapentin, nefopam, paracetamol, pentazocine, pregabalin, sumatriptan, and non-steroidal anti-inflammatory drugs (NSAIDs) including salicylates such as aspirin, methyl salicylate and difunisal, arylalkanoic acids such as indomethacin, sulindac and diclofenac, 2-arylpropionic acids (profens), such as ibuprofen, ketoprofen, naproxen, ketorolac, etodolac, carprofen and feniprofen, N-arylanthranilic acids (fenamic acids), such as mefenamic acid and tolfenamic acid, oxicams such as piroxicam and meloxicam, coxibs such as celecoxib, rofecoxib, valdecoxib, parecoxib and etoricoxib, and sulphonanilides such as nimesulide.

Anti-migraine agents include, for example, 5-HT antagonists (such as methysergide, cyproheptadine, pisorifen), 5-HT agonists (such as sumpatriptan, zolmitriptan, naratriptan, rizatriptan, almotriptan, eletriptan, and frovatriptan), ergot alkaloids and their derivatives (such as ergotamine and dihydroergotamine), adrenoceptor agonists (such as clonodine and propanalol) and calcium antagonists (such as dihydropyridines and verapamil).

In one embodiment of the present invention, the analgesic agent is not an opioid or is not fentanyl.

Mixtures of analgesic agents, including mixtures of agents from two or more of the aforementioned categories, may be included in the compositions of the present invention.

Mixtures of analgesic agents and one or more further therapeutically active agents are also envisaged, especially wherein the further therapeutically active agent is an anti-emetic agent.

The coatings and/or matrix excipients that are included in the compositions of the present invention will be selected to complement the analgesic agent(s) also included, and must be chosen for their ability to control the release of the analgesic agent(s) to provide the desired release profile. This will involve providing a rapid therapeutic effect, as well as a sustained period of therapeutic effect, as well as providing desired local and/or plasma concentrations.

In a preferred embodiment of the present invention, in order to provide the rapid effect following administration, the composition comprises a form of the analgesic agent which is readily soluble in an aqueous environment such as that to which the composition is exposed to following oral administration, and/or a form which is quickly absorbed via the gastrointestinal tract into the bloodstream.

Various known techniques may be used to prepare analgesic agents in forms which are more readily dissolved in an aqueous environment. Examples of such techniques are disclosed in WO 2004/011537 and WO 2005/073296, WO 2005/07330, WO 2005/075546 and WO 2005/075547.

The techniques disclosed in the prior art involve solubilising otherwise poorly soluble active agents and they are useful because they enable compositions to be formulated with otherwise “difficult” agents, such as those in their base form which are normally only poorly soluble. Normally, the poor solubility of the base form of some pharmaceutically active agents severely restricts their use, with salt forms being employed instead. However, the base form often exhibits faster onset of the therapeutic effect and, in some cases, better absorption.

Thus, in a preferred embodiment of the present invention, the compositions comprise the base form of the analgesic agent. Where necessary, a solubilisation technique is used, in order to enhance the solubility of the base form. In a particularly preferred embodiment, the analgesic agent is an opioid base, such as oxycodone base, oxymorphone base, hydrocodone base, hydromorphone base, bupromorphine base or morphine base.

Alternatively or additionally, the agent may be provided in the form of nano-sized particles. This will also enhance solubilisation and absorption. Known techniques from the art may be used to produce these ultra-fine particles.

In a further embodiment of the present invention, the composition includes a therapeutically active agent other than the analgesic agent. Such therapeutically active agents are preferably selected to have an effect which complements that of the analgesic agent or which will assist the intended purpose of the composition. The therapeutically active agent may be known for use in treating the underlying cause of the condition to be treated using the composition and/or it may treat the symptoms of that condition or a related condition. It is also possible for the further therapeutic agent to be included in the compositions of the present invention in order to treat the side effects of the analgesic agent. For example, the further therapeutic agent may be an anti-emetic, especially where the analgesic agent is an opioid.

The compositions according to the present invention may be used to provide improved treatment of specific pain or pain-related conditions by combining analgesic agents with particular desirable properties and orchestrating their release from the composition to control their plasma concentration over time. This control of the strength and timing of analgesic effects provides tailored treatment for particular conditions, examples of which are discussed in detail below. A particular benefit of compositions providing tailored analgesic is that it can mean that pain can be treated very efficiently, allowing milder analgesic agents to be used, rather than having to resort to stronger agents, which are often associated with more severe or more undesirable side effects.

Some embodiments of the present invention which are of particular interest are described below.

Migraine is a common and unpleasant condition, the causation of which is not well understood. One commonly held view is that vascular changes are responsible, and that these are triggered by 5-HT release. The most common pattern of events in a migraine attack consists of an initial visual disturbance (the aura), in which the central area of the visual field is lost, and the surrounding area displays a jagged, flickering pattern. This visual disturbance is followed about 30 minutes later by a severe headache, often with photophobia, nausea and vomiting, which lasts for several hours.

Despite the fact that there is much disagreement relating to the cause and underlying mechanisms associated with the onset and progression of a migraine, a large number of therapeutic compositions have been found that treat the condition, with varying degrees of success. For example 5-HT antagonists (such as methysergide, cyproheptadine, pisorifen), 5-HT agonists (such as sumpatriptan, zolmitriptan, naratriptan, rizatriptan, almotriptan, eletriptan, and frovatriptan), ergot alkaloids and their derivatives (such as ergotamine and dihydroergotamine), adrenoceptor agonists (such as clonodine and propanalol) and calcium antagonists (such as dihydropyridines and verapamil) have all proven to be useful in the treatment of migraine.

Unfortunately, however, subjects being treated with the aforementioned conventional therapeutic compositions experience a wide range of side effects. Perhaps the most unpleasant of these side effects are re-bound headaches. Re-bound headaches often occur within one or two days following the treatment of migraine with one of the abovementioned compositions. More than one re-bound headache may be experienced over a number of days. Migraine sufferers that require frequent treatment with one of the conventional migraine therapeutic compositions most commonly experience re-bound headaches. Accordingly, subjects are normally advised not to take more than 2 to 3 doses of the conventional compositions over the course of a week. Such sufferers who need to take more than 2 to 3 doses of these compositions in a week can, therefore, get into a cycle of migraine headaches and re-bound headaches. Often such suffers are forced to stop treatment with the anti-migraine therapeutic compositions, and therefore suffer the symptoms of migraine, in order to break free from the aforementioned cycle. Additionally, the onset of re-bound headaches has been found to increase the anxiety levels of migraine sufferers because of the stress of coping with the pain, and because many migraine sufferers interpret the re-bound headache as the start of a migraine. Many believe that this increase in anxiety can induce further migraines.

Thus, it is a further object of the present invention to develop compositions that can treat both migraines and the associated re-bound headaches.

According to a further aspect of the present invention, there is provided a pharmaceutical composition comprising an anti-migraine agent and an analgesic agent, wherein the composition is prepared for separate, sequential or simultaneous administration of the anti-migraine agent and the analgesic agent.

In a preferred embodiment, the anti-migraine agent is released from the composition as quickly as possible and has a rapid onset of effect. This allows the migraine to be instantly treated or even prevented. The analgesic agent is preferably released from the composition in a controlled manner, preferably after an initial delay. This allows the analgesic agent to provide relief from the pain of re-bound headaches over a period of 1, 2 or 3 days after the initial migraine.

The anti-migraine agent can be any agent that is capable of treating or attenuating the symptoms of migraine. The anti-migraine agent may be a 5-HT antagonist, such as methysergide, cyproheptadine or pisorifen. The anti-migraine agent may be a 5-HT agonist, such as sumatriptan, zolmitriptan, naratriptan, rizatriptan, almotriptan, eletriptan or frovatriptan. The anti-migraine agent may be an ergot alkaloid, or its derivatives, such as ergotamine or dihydroergotamine. The anti-migraine agent may be an adrenoceptor agonist, such as clonodine or propanalol. The anti-migraine agent may be a calcium antagonist, such as dihydropyridines or verapamil. Preferably, the anti-migraine agent is one that is available without a prescription. Preferably, the anti-migraine agent is a triptan, preferably sumatriptan.

The analgesic agent is preferably any agent that is capable of treating or attenuating a re-bound headache brought on by a treatment involving the use of an anti-migraine agent. The analgesic agent may be a non-steroidal anti-inflammatory drug (NSAID). The analgesic agent may be a salicylic acid, such as aspirin, diflunisal or benorylate. The analgesic agent may be a propionic acid, such as naproxen, ibuprofen, flurbiprofen, fenbufen or ketoprofen. The analgesic agent may be an acetic acid, such as indomethacin or sulindac. The analgesic agent may be a fenamate, such as meclofenamic acid or mefanimic acid. The analgesic agent may be an oxicam, such as piroxicam or tenoxicam. The analgesic agent may be a pyrazolone, such as phenylbutazone or azapropazone. The analgesic agent may be tolmetin or paracetamol. Preferably, the analgesic agent is available without a prescription, such as aspirin, paracetamol or ibuprofen. Preferably, the analgesic agent is ibuprofen.

The analgesic agent can be administered before, after or at the same time as the anti-migraine agent, or a combination thereof.

In one embodiment where the analgesic agent and the anti-migraine agent are ingested at the same time, the analgesic agent and the anti-migraine agent are formulated together in a single composition that is prepared so as to administer the analgesic agent before, after, at the same time or a combination thereof, as the anti-migraine agent in the gastrointestinal tract of the subject to be treated. It is preferred that the anti-migraine agent achieves a peak plasma concentration rapidly after ingestion. In this way the migraine may be most efficiently treated before the symptoms are given time to fully develop. Accordingly, in an embodiment of the invention, the anti-migraine agent achieves a peak plasma concentration within less than 30, 15, 10, 5 or 1 minute of ingestion. It is also preferred that the analgesic agent is not administered until after the migraine has gone, but before the re-bound headache begins. However, when the analgesic agent is administered, it is preferred that this administration is over an extended period of time.

The release of one or both of the agents may provide spikes in the plasma concentration, with a rapid increase and then rapid decrease in the concentration, thereby producing peaks followed by troughs of the plasma concentration of the agent in the subject being treated.

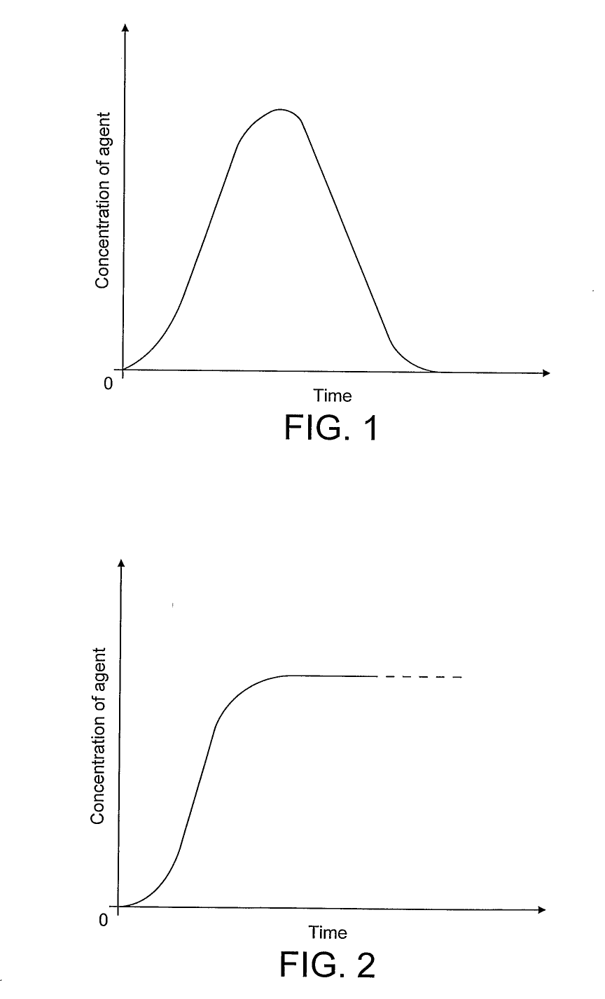

FIG. 1 is a schematic graph showing an example of a release profile with a concentration spike of an agent. Alternatively, administration of one or both of the agents may be such that the plasma concentration of the agent rises to a plateau (i.e. a relatively constant administration being maintained over a period of time), an example of which is shown in

FIG. 2.

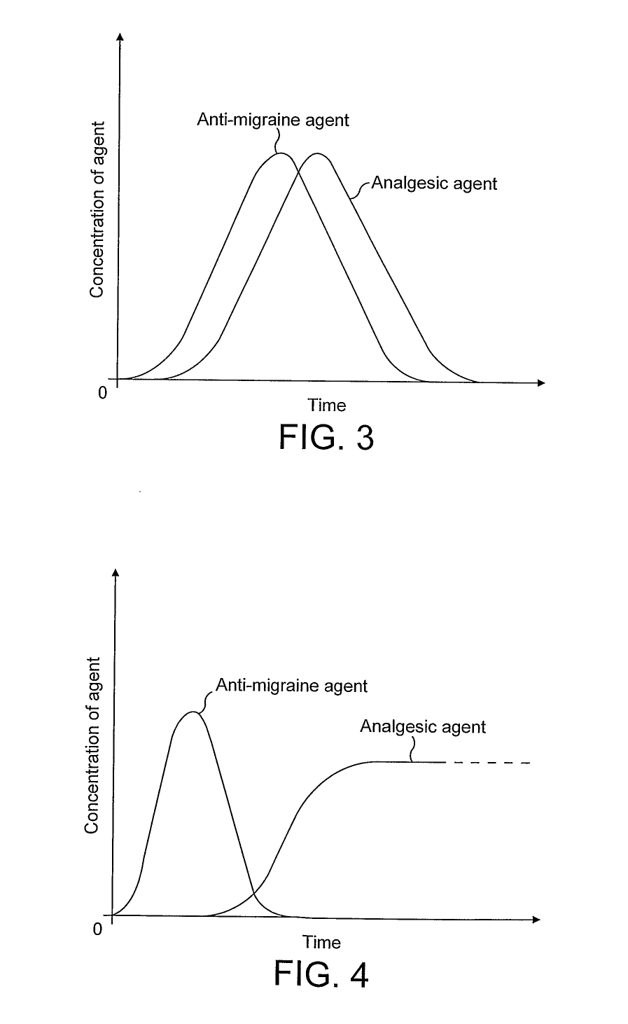

FIG. 3 shows a release profile for an embodiment of the invention where the composition releases the anti-migraine agent to provide a spiked plasma concentration, followed by a spiked plasma concentration of the analgesic agent.

FIG. 4 shows a release profile for an embodiment where the anti-migraine agent is administered to provide a spiked concentration, followed by a constant concentration of the analgesic agent.

Accordingly, in one embodiment of the present invention, the anti-migraine agent achieves a peak plasma concentration in the subject to be treated, followed by a subsequent peak in the plasma concentration of the analgesic agent in the subject.

In an alternative embodiment of the present invention, the anti-migraine agent achieves a peak plasma concentration in the subject to be treated, followed by a rise in the plasma concentration of the analgesic agent which then plateaus.

It has been found that the delay between achieving the peak plasma concentration of the anti-migraine agent and that of the analgesic agent, or the delay between achieving a peak plasma concentration of the anti-migraine agent and achieving a plateaued concentration of the analgesic agent should be selected so as to be shorter than the usual delay between the onset of a migraine and the onset of a re-bound headache. Not wishing to be restricted further, but in the interest of clarity, the delay may be between 1 and 48, 6 and 36 or 12 and 24 hours.

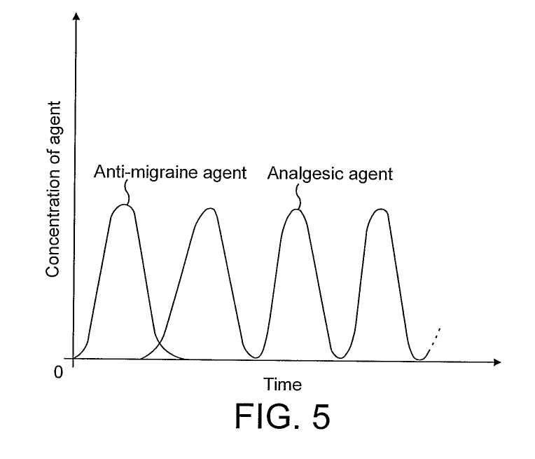

In some embodiments the spiked administration of either or both agents may be repeated, in a cyclical manner, thereby achieving repeated peak plasma concentrations for that agent. In some embodiments, for example, a peak plasma concentration is achieved for the anti-migraine agent, which is followed by repeated peak plasma concentrations for the analgesic agent (e.g. see FIG. 5).

In the case of the analgesic agent, the numbers of repeated peak plasma concentrations of the analgesic agent should be selected so as to ensure as consistent an analgesic effect as possible, and so is dependent on the specific analgesic agent used, and more specifically, is dependent on the length of time the analgesic agent will remain actively present in the plasma of the subject being treated. Preferably, a peak plasma concentration of the analgesic agent occurs 1, 2, 3, 4, 5, 6, 7, 8, 9, 10, 11, 12, 13, 14, 15, 16, 17, 18, 19, 20, 21, 22, or 24 times a day.

In one embodiment only one peak plasma concentration of the anti-migraine agent occurs.

In some embodiments, either or both agents are administered constantly over a period of time, thereby achieving a plateau of the plasma concentration for that agent. For example, a single peak plasma concentration of the anti-migraine agent may be followed by a plateau of the plasma concentration of the analgesic agent (e.g. see FIG. 4).

In the case of the anti-migraine agent, the period of repeated peak plasma concentrations of the anti-migraine agent, or the period of the plateaued plasma concentration of the anti-migraine agent should be selected to coincide with the period in which the subject suffers the migraine. Preferably, this period is 1, 2, 3, 4, 5, 6, 7, 8, 9, 10, 12, 13, 14, 15, 16, 17, 18, 19, 20, 21, 22, 23 or 24 hours.

In the case of the analgesic agent, the period of repeated peak plasma concentrations of the analgesic agent, or the period of the plateaued plasma concentration of the analgesic agent should be selected to coincide with the period in which the subject suffers re-bound headache. Preferably, this period is 6, 12, 18, 24, 30, 36, 42, 48, 52, 58, 64, 70, 76, 82, 90 or 96 hours.

It is difficult to maintain such regularity and frequency of dosing by controlling the ingestion of the agents, particularly if the subject to be treated is an infant or an elderly person. Such regimens are also inconvenient to maintain, and it is easy to miss a timed dose by forgetting. Additionally, it is difficult to ingest multiple doses spread equally over a 24-hour period without disrupting sleep. Hence, even if the subject being treated can stick to the strict and complicated regimens suggested above, ingested doses are usually only spread evenly throughout the subject's waking hours. The gap in administration of, for example, the analgesic agent during sleep time can mean that the plasma concentration of the analgesic agent drops below an effective level, and so the subject being treated may be wakened from their sleep by the pain of a headache.

Accordingly, in a preferred embodiment of the present invention, the composition provides relief from migraine and/or re-bound headaches for a period of at least 8 hours, preferably at least 12 hours, at least 24 hours, at least 48 hours or more.

In one embodiment, such compositions can be prepared as two separate dosage forms, one comprising the anti-migraine agent, and the other comprising the analgesic agent. These separate dosage forms are preferably administered at the same time or one immediately after the other.

In an alternative embodiment, the compositions are provided as a single dosage form comprising both the anti-migraine agent and the analgesic agent.

The compositions of the present invention are also particularly suited to the efficient and effective treatment of pain and of pain-related conditions. For example, the compositions may be used for the treatment of pain-related sleep disturbance, for example in subjects with chronic neuropathic pain, such as that associated with fibromyalgia, arthritis or cancer.

In an embodiment of the present invention, the compositions comprise a first analgesic agent which rapidly released, and a second analgesic agent the release of which is controlled to provide a therapeutic effect until at least 6 hours after administration, preferably at least 8 hours.

Preferably, the first analgesic agent to causes drowsiness, so that it encourages the patient to sleep, not only by quickly and effectively relieving pain, but by also causing drowsiness.

In some embodiments, the therapeutic effect of the second analgesic agent has a delayed onset, so that the second analgesic agent does not reach therapeutic concentrations until the plasma concentration of the first analgesic agent has started to decline.

Preferably, the first analgesic agent is a tricyclic antidepressant, such as amitriptyline, and the second analgesic agent is a GABA agonist, for example gabapentin. This combination of analgesic agents provides an effective combination of effects. Upon administration, the subject feels an initial pain relief and drowsiness which helps him or her to fall asleep. Then, the sustained analgesia from the second agent ensures that the subject is not awoken by pain during the night. Gabapentin is particularly suited for this purpose, as it has been found that it can increase slow-wave sleep in adults.

The synergistic combination of the two analgesics proposed in this embodiment allow effective use of analgesic agents which are generally used to treat pain classified as mild or mild to moderate according to the World Health Organisation's “analgesic ladder”. Where pain-related sleep disturbance is treated using normal analgesic compositions, patients and practitioners are likely to resort to stronger analgesics to achieve an equivalent effect, such as opioids and the like.

When used for such a purpose, the composition is preferably administered once a day, prior to bedtime. The composition so administered is also preferably in the form of multiparticulates, as discussed above, to avoid any discomfort that may arise from swallowing an oral solid dosage from shortly before lying down.

1. A pharmaceutical composition comprising one or more analgesic agents, wherein the release of said agent(s) is controlled to provide a rapid onset of analgesic effect following administration, and preferably also delayed and/or sustained release providing an extended period of analgesia.

2. A composition as claimed in claim 1, wherein the composition is for oral administration.

3. A composition as claimed in claim 1, wherein the composition provides an analgesic effect for a period of at least about 8 hours.

4. A composition as claimed in claim 1, wherein the analgesic agent is included in its base form.

5. A composition as claimed in claim 1, wherein the composition is for gastrointestinal deposition and comprises a non-compressed free-flowing plurality of particles.

6. A composition as claimed in claim 5, wherein the particles have a mean diameter of greater than 10 μm to about 1 mm.

7. A composition as claimed in claim 5, further comprising a water-soluble excipient, the composition being capable of dissolving or dispersing in a subject's mouth within 1 minute after administration, preferably without the co-administration of a fluid.

8. A composition as claimed in claim 1, wherein the composition comprises two or more analgesic agents.

9. A composition as claimed in claim 8, wherein a first analgesic agent is rapidly released and a second analgesic agent is released slowly over a period of at least 8 hours.

10. A composition as claimed in claim 9, wherein the first analgesic agent provides a therapeutically effective plasma concentration within a period of no more than 30 minutes following administration of the composition.

11. A composition as claimed in claim 9, wherein the first analgesic agent is an anti-migraine agent.

12. A composition as claimed in claim 11, wherein the anti-migraine agent is a 5-HT antagonist, a 5-HT agonist, an ergot alkaloid, or its derivative, an adrenoceptor agonist, a calcium antagonist or a combination thereof.

13. A composition as claimed in claim 11, wherein the anti-migraine agent is sumatriptan, preferably sumatriptan base.

14. A composition as claimed in claim 11, wherein the second analgesic agent is a non-steroidal anti-inflammatory drug.

15. A composition as claimed in claim 14, wherein the second analgesic agent is a salicylic acid, a propionic acid, an acetic acid, a fenamate, an oxicam, a pyrazolone, paracetamol, tolmetin or a mixture thereof.

16. A composition as claimed in claim 15, wherein the analgesic agent is ibuprofen.

17. A composition as claimed in claim 11, for treating migraine.

18. A composition as claimed in claim 9, wherein the first analgesic agent is a tricyclic antidepressant and the second analgesic agent is a GABA agonist.

19. A composition as claimed in claim 9, wherein the first analgesic agent is amitriptyline and the second analgesic agent is gabapentin.

20. A composition as claimed in claim 1, wherein the composition is for treating pain-related sleep disturbance.

21. A composition as claimed in claim 1, wherein the composition provides an analgesic effect for a period of at least about 12 hours.

22. A composition as claimed in claim 1, wherein the composition provides an analgesic effect for a period of at least about 24 hours.
